(19) United States
(12) Patent Application Publication
(10) Pub. No.: US 2002/0103249 A1
(43) Pub. Date: Aug. 1, 2002

(54) Combination of a serotonin reuptake inhibitor and irindalone

(75) Inventors: Klaus Peter Bogeso, Horsholm (DK); Thomas Ivo Franciscus Hubert Cremers, Groningen (NL)

Correspondence Address: DARBY & DARBY P.C., 805 Third Avenue, New York, NY, 10022, US

(73) Assignee: H. Lundbeck A/S

(21) Appl. No.: 09/731,411

(22) Filed: Dec. 6, 2000

Related U.S. Application Data
(60) Provisional application No. 60/169,245, filed on Dec. 6, 1999.

(51) Int. Cl. A61K 31/497

(52) U.S. Cl. 514/419

(57) ABSTRACT

The present invention relates to the use of a combination of irindalone and a serotonin reuptake inhibitor (SRIs), or any other compound, which causes an elevation in the level of extracellular serotonin, for the treatment of depression and other affective disorders.

The present invention relates to the use of a combination of irindalone and a serotonin reuptake inhibitor (SRI), or any other compound, which causes an elevation in the level of extracellular serotonin, for the treatment of depression and other affective disorders.

### BACKGROUND

Selective serotonin reuptake inhibitors (hereinafter referred to as SSRIs) haste become first choice therapeutics in the treatment of depression, certain forms of anxiety and social phobias, because they are effective, well tolerated and have a favourable safety profile compared to the classic tricyclic antidepressants.

However, clinical studies on depression and anxiety disorders indicate that non-response to SSRIs is substantial, up to 30%. Another, often neglected, factor in antidepressant treatment is compliance, which has a rather profound effect on the patient's motivation to continue pharmacotherapy.

First of all, there is the delay in therapeutic effect of SSRIs. Sometimes symptoms even worsen during the first weeks of treatment. Secondly, sexual dysfunction is a side effect common to all SSRIs. Without addressing these problems, real progress in the pharmacotherapy of depression and anxiety disorders is not likely to happen.

In order to cope with non-response, psychiatrists sometimes make use of augmentation strategies. Augmentation of antidepressant therapy may be accomplished through the co-administration of mood stabilizers such as lithium carbonate or triiodothyronin or by the use of electroshock.

In 1993, an augmentation strategy with pindolol was described by Artigas et al. in Trends Pharmacol. Sci. 1993, 14, p 262-263. Artigas' idea is based on intracerebral microdialysis experiments in animals. In fact, later neurochemical studies built on the desensitization hypothesis by Blier and co-workers stated that the delay in therapeutic effect of antidepressants is related to a gradual desensitization of 5-HT autoreceptors (Blier et al. J. Clin. Psycipharomacol. 1987, 7 suppl. 6, 24S-35S). A key point in their hypothesis is that the effects of SSRIs on the release-controlling somatodendritic autoreceptors (5-HT_{1A}) limit the release of 5-HT in terminal areas and thus the effect of 5-HT uptake inhibition in those regions. This is supported by microdialysis experiments in rats, showing that the increase in extracellular 5-HT elicited by a single dose of an SSRI is augmented by co-administration of a 5-HT_{1A} autoreceptor antagonist (Invernizzi et al. Brain Res. 1992, 584, p 322-324 and Hjorth, S., J. Neurochem, 1993, 60, p 776-779).

The effect of combined administration of a compound that inhibits serotonin reuptake and a 5-HT_{1A} receptor antagonist has been evaluated in several studies (Innis, R. B. et al. Eur. J. Pharmacol. 1987, 143, p. 1095-204 and Gartside, S. E., Br. J. Pharmacol, 1995, 115, p 1064-1070, Blier, P. et al. Trends in Pharmacol. Science 1994, 15, 220). In these studies it was found that 5-HT_{1A} receptor antagonists would abolish the initial brake on 5-HT neurotransmission induced by the serotonin reuptake inhibitors and thus produce an immediate boost of 5-HT transmission and a rapid onset of therapeutic action.

Several patent applications have been filed which cover the use of a combination of a 5-HT_{1A} antagonist and a serotonin reuptake inhibitor for the treatment of depression (see e.g. EP-687 472 and EP-A2-714 663).

Another approach to increase terminal 5-HT would be through blockade of the 5-HT_{1B} autoreceptor. Microdialysis experiments in rats have indeed shown that increase of hippocampal 5-HT by citalopram is potentiated by GMC 2-29, an experimental 5-HT_{1B} receptor antagonist.

Several patent applications covering the combination of an SSRI and a 5-HT_{1B} antagonist or partial agonist have also been filed (WO 97/28141, WO 96/03400, EP-A-701819 and WO 99/13877).

It has now surprisingly been found that irindalone having the general formula

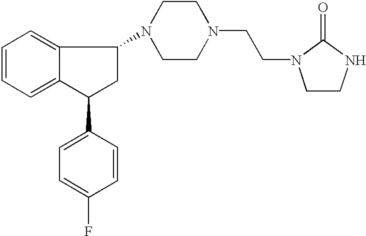

or pharmaceutically acceptable salts thereof may be used to augment and provide faster onset of the therapeutic effect of serotonin reuptake inhibitors.

The invention

The present invention thus provides:

The present invention relates to the use of irindalone or a pharmaceutically acceptable salt thereof for the preparation of a pharmaceutical composition to be used in combination with a serotonin reuptake inhibitor or any other compound, which causes an elevation in the level of extracellular serotonin.

In particular, the present invention relates to the use of irindalone or a pharmaceutically acceptable salt thereof for the preparation of a pharmaceutical composition useful for augmenting and/or providing faster onset of the therapeutic effect of a serotonin reuptake inhibitor or any other compound, which causes an elevation in the level of extracellular serotonin.

More particularly, the present invention relates to the use as above, of irindalone, or a pharmaceutically acceptable salt thereof for the treatment of depression, anxiety disorders and other affective disorders, eating disorders such as bulimia, anorexia and obesity, phobias, dysthymia, premenstrual syndrome, cognitive disorders, impulse control disorders, attention deficit hyperactivity disorder and drug abuse, in particular depression with a serotonin reuptake inhibitor or any other compound, which causes an elevation in the level of extracellular serotonin.

The anxiety disorders mentioned above include general anxiety disorder, panic anxiety, obsessive compulsive disorder, acute stress disorder, post trauma stress disorder or social anxiety disorder.

As used herein, the term augmenting covers improving the therapeutic effect and/or potentiating the therapeutic effect of an SRI or a compound which causes an elevation in the level of extracellular 5-HT.

In a further embodiment, the invention relates to the use of irindalone or a pharmaceutically acceptable salt thereof and a compound, which is a serotonin reuptake inhibitor, or a compound, which causes an elevation in the level of extracellular serotonin, for the preparation of a pharmaceutical composition or kit for the treatment of diseases or disorders responsive to the therapeutic effect of a serotonin reuptake inhibitor, or any other compound, which causes an elevation in the level of extracellular serotonin.

The diseases responsive to a serotonin reuptake inhibitor include depression, anxiety disorders and other affective disorders, eating disorders such as bulimia, anorexia and obesity, phobias, dysthymia, premenstrual syndrome, cognitive disorders, impulse control disorders, attention deficit hyperactivity disorder and drug abuse, in particular depression.

The term anxiety disorders is as defined above.

In one embodiment, the present invention relates to the use of irindalone or a pharmaceutically acceptable salt thereof for the preparation of a pharmaceutical composition as above, which is adapted for simultaneous administration of the active ingredients. In particular, such pharmaceutical compositions may contain the active ingredients within the same unit dosage form, e.g. in the same tablet or capsule. Such unit dosage forms may contain the active ingredients as a homogenous mixture or in separate compartments of the unit dosage form.

In another embodiment, the present invention relates to the use of irindalone or a pharmaceutically acceptable salt thereof for the preparation of a pharmaceutical composition or kit as above, which is adapted for sequential administration of the active ingredients. In particular, such pharmaceutical compositions may contain the active ingredients in discrete unit dosage form, e.g. discrete tablets or capsules containing either of the active ingredients. These discrete unit dosage forms may be contained in the same container or package, e.g. a blister pack.

As used herein The term kit means a pharmaceutical composition containing each of the active ingredients, but in discrete unit dosage forms.

The invention also relates to a pharmaceutical composition or kit comprising irindalone or a pharmaceutically acceptable salt thereof and a compound, which is a serotonin reuptake inhibitor, or any other compound, which causes an elevation in extracellular 5-HT, and optionally pharmaceutically acceptable carriers or diluents.

The pharmaceutical composition or kit of the invention may be adapted for simultaneous administration of the active ingredients or for sequential administration of the active ingredients, as described above.

Finally, the present invention relates to a method for the treatment of diseases or disorders responsive to a serotonin reuptake inhibitor or any other compound, which causes an elevation in the level of extracellular serotonin, comprising administering irindalone or a pharmaceutically acceptable salt thereof and a serotonin reuptake inhibitor, or a compound, which causes an elevation in the level extracellular serotonin, to an individual in need thereof,

In particular, the present invention relates to a method for augmenting and/or providing faster onset of the therapeutic effect of a serotonin reuptake inhibitor or any other compound, which causes an elevation in the level extracellular serotonin, comprising administering irindalone or a pharmaceutically acceptable salt thereof to an individual to be treated with or undergoing treatment with the serotonin reuptake inhibitor or any other compound, which causes an elevation in the level of extracellular serotonin.

The individuals, which may benefit from treatment with a combination as above, may suffer from depression, anxiety disorders and other affective disorders, eating disorders such as bulimia, anorexia and obesity, phobias, premenstrual syndrome, dysthymia, cognitive disorders, impulse control disorders, attention deficit hyperactivity disorder and drug abuse, in particular depression.

As mentioned above, anxiety disorder includes general anxiety disorder, panic anxiety, obsessive compulsive disorder, acute stress disorder, post trauma stress disorder or social anxiety disorder.

Irindalone and the serotonin reuptake inhibitor may be administered simultaneously as described above.

Alternatively, the active ingredients may be administered sequentially, e.g. in two discrete unit dosage forms as described above.

Irindalone and pharmaceutically acceptable salts thereof having the general formula

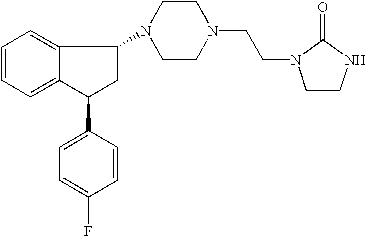

are covered by EP-B1-183 349. The compounds claimed therein are described as being useful for the treatment of hypertension and other cardiovascular diseases as well as anxiety.

Irindalone has also been described as a potent peripheral 5-HT₂ antagonist with no or only low affinity for 5-HT_{1A} and 5-HT_{1B} receptors (1-Hyttel et al., Drug Dev. Res. 1988b, 15, 389-404, Arnt et al., Drug Dev. Res. 1989, 16, 59-70 and Bøgesø et al., Schizophrenia, Alfred Benzon Symposium 38, 1995, p. 361-374).

It has now, surprisingly, been found that co-administration of irindalone and a serotonin reuptake inhibitor produces a significant increase in the level of serotonin in terminal areas, as measured in microdialysis experiments, compared to the administration of the serotonin reuptake inhibitor alone. Administration of irindalone alone causes no increase in serotonin levels as measured in microdialysis experiments.

As mentioned above, serotonin reuptake inhibitors show delayed onset of action. Even in responders to SSRIs, several weeks of treatment are necessary to achieve a relief in symptoms. Animal studies have shown that irindalone may provide fast onset of therapeutic effect of serotonin reuptake inhibitors.

It has also been found that irindalone potentiates the anxiolytic potential of serotonin reuptake inhibitors.

The use of a combination of irindalone and a serotonin reuptake inhibitor may greatly reduce the amount of serotonin reuptake inhibitor necessary to treat depression and other affective disorders and may thus reduce the side effects caused by the serotonin reuptake inhibitor. In particular, the combination of a reduced amount of SRI and irindalone may reduce the risk of SSRI-induced sexual dysfunction and sleep disturbances.

Co-administration of irindalone and a serotonin reuptake inhibitor may also be useful for the treatment of refractory depression, i.e. depression, which cannot be treated appropriately by administration of a serotonin reuptake inhibitor alone. Typically, irindalone may be used as add-on therapy for the augmentation of the response to SRIs in patients where at least 40-60% reduction in symptoms has not been achieved during the first 6 weeks of treatment with an SRI.

Many antidepressants with serotonin reuptake inhibiting effect have been described in the literature. Any pharmacologically active compound, which primarily or partly exert its therapeutic effect via inhibition of serotonin reuptake in the CNS, may benefit from augmentation with irindalone.

The following list contains a number of serotonin reuptake inhibitors, which may benefit from augmentation with irindalone: citalopram, escitalopram, fluoxetine, R-fluoxetine, sertraline, paroxetine, fluvoxamine, venlafaxine, duloxetine, dapoxetine, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, WY 27587, WY 27866, imeldine, ifoxetine, tiflucarbine, viqualine, milnacipran, bazinaprine, YM 922, S 33005, F 98214-TA, OPC 14523, alaproclate, cyanodothepine, trimipramine, quinupramine, dothiepin, amoxapine, nitroxazepine, McN 5652, McN 5707, O1 77, Org 6582, Org 6997, Org 6906, amitriptyline, amitriptyline N-oxide, nortriptyline, CL 255.663, pirlindole, indatraline, LY 113.821, LY 214.281, CGP 6085 A, RU 25.591, napamezole, diclofensine, trazodone, EMD 68.843, BMY 42.569, NS 2389, sercloremine, nitroquipazine, ademethionine, sibutramine and clovoxamine. The compounds mentioned above may be used in the form of the base or a pharmaceutically acceptable acid addition salt thereof.

Other therapeutic compounds, which may benefit from augmentation with irindalone, include compounds, which causes an elevation in the extracellular level of 5-HT in the synaptic cleft, although they are not serotonin reuptake inhibitors. One such compound is tianeptine.

The above list of serotonin reuptake inhibitors and other compounds, which causes an increase in the extracellular level of serotonin, may not be construed as limiting.

SRIs, which are particularly preferred according to the present invention, include citalopram, escitalopram, fluoxetine, sertraline, paroxetine, fluvoxamine, venlafaxine, dapoxetine, nefazodone, imipramin, femoxetine and clomipramine.

The term selective serotonin reuptake inhibitor (SSRI) means an inhibitor of the monoamine transporters which has stronger inhibitory effect at the serotonin transporter than the dopamine and the noradrenaline transporters.

Selective serotonin reuptake inhibitors (SSRIs) are among the most preferred serotonin reuptake inhibitors used according to the present invention. Particularly preferred SSRIs according to the invention are citalopram, escitalopram, fluoxetine, fluvoxamine, sertraline or paroxetine.

The active ingredients according to the invention, i.e. irindalone and the SRI or a compound causing an increase in extracellular serotonin levels, may be used in the free base form or in the form of a pharmaceutically acceptable acid addition salt thereof, the latter being obtainable by reaction of the base form with an appropriate acid.

Citalopram is preferably used in the form of the hydrobromide or as the base, escitalopram in the form, of the oxalate, fluoxetine, sertraline and paroxetine in the form of the hydrochloride and fluvoxamine in the form of the maleate.

Irindalone is preferably used in the form of the tartrate salt.

As mentioned above, the combination of irindalone with a serotonin reuptake inhibitor unexpectedly shows a synergistic effect on the central nervous system (CNS). As a consequence, combination therapy using irindalone and lower doses of the serotonin reuptake inhibitor than normally used in monotherapy, may be effective, and side-effects associated with the larger amounts of serotonin reuptake inhibitor used in monotherapy may be reduced or prevented altogether.

Additionally, combination therapy with irindalone using a normal dose of serotonin reuptake inhibitor has the advantage that an effective CNS effect may be obtained in the often large number of patients who do not respond to conventional monotherapy with SSRIs.

The amount of irindalone used in combination therapy may range from about 0.1 to about 150 mg/day, particularly from about 0.1 to about 100 mg/day and more particularly from about 0.5 to about 50 mg/day and even more particularly from about 1 to about 5 mg/day.

Serotonin reuptake inhibitors, including the SSRIs specifically mentioned hereinabove, differ both in molecular weight and in activity. As a consequence, the amount of serotonin reuptake inhibitor used in combination therapy depends on the nature of said serotonin reuptake inhibitor. In one embodiment of the invention, the serotonin reuptake inhibitor or the compound causing an increase in the level of extracellular 5-HT, is administered at lower doses than required when the compound is used alone. In another embodiment, the serotonin reuptake inhibitor or the compound causing an increase in the level of extracellular 5-HT, is administered in normal doses.

To prepare the pharmaceutical compositions of this invention, an appropriate amount of the active ingredient(s), in salt form or base form, is combined in an intimate admixture with a pharmaceutically acceptable carrier, which can take a wide variety of forms depending on the form of preparation desired for administration. These pharmaceutical compositions are desirably in unitary dosage form suitable for administration orally, rectally, percutaneously or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed.

It is especially advantageous to formulate the aforementioned pharmaceutical compositions in dosage unit form for ease of administration and uniformity of dosage. As used in the specification and claims, unit dosage form refers to physically discrete units suitable as unitary dosages, each unit containing a predetermined quantity of active ingredient(s) calculated to produce the desired therapeutic effect, in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

Irindalone may be administered before, during or after the administration of the serotonin reuptake inhibitor provided that the time between the administration of irindalone and the administration of the serotonin reuptake inhibitor is such that ingredients are allowed to act synergistically on the CNS. When simultaneous administration of irindalone and a serotonin reuptake inhibitor is envisaged, a composition containing both a serotonin reuptake inhibitor and irindalone may be particularly convenient. Or, irindalone and the serotonin reuptake inhibitor may be administered separately in the form of suitable compositions. The compositions may be prepared as described hereinabove.

The present invention also comprises products containing irindalone and a serotonin reuptake inhibitor as a combination preparation for simultaneous, separate or sequential use in psychiatric drug therapy. Such products may comprise, for example, a kit comprising discrete unit dosage forms containing irindalone and discrete unit dosage forms containing a serotonin reuptake inhibitor, all contained in the same container or pack, e.g. a blister pack.

The above mentioned preparations for simultaneous or sequential administration may instead of a serotonin reuptake inhibitor contain another compound causing an elevation in the level of extracellular 5-HT.

### EXPERIMENTALS

Microdialysis experiments in conscious rats.

Treatment and drugs

Male Sprague-Dawley rats, initially weighing 250-300 g, were used. The animals were housed under a 12-hr light/dark cycle under controlled conditions for regular in-door temperature (21±2° C.) and humidity (55±5%) with food and tap water available ad libitum. For treatments with osmotic mini pumps Alzet, 2ML2 and 2ML4 (Alza Corporation, Palo Alto, USA) were used. The pumps were filled under aseptic conditions and implanted subcutaneously under Sevorane anaesthesia (Abbott Laboratories, Chicago, USA)

Single injections:

Paroxetine and irindalone were injected alone or contemporary in doses of 5 mg/kg and 1 mg/kg s.c., respectively.

Combined treatment with paroxetine and irindalone on extracellular 5-HT after acute [2 days] and chronic [14 days] treatments with mini pumps:

Groups:

1. Vehicle, 2 days

2. Paroxetine (10 mg/kg/day), 2 days

3. Paroxetine (10 mg/kg/day)+irindalone (5 mg/kg/day), 2 days

4. Vehicle, 14 days

5. Paroxetine (10 mg/kg/day), 14 days

6. Paroxetine (10 mg/kg/day)+irindalone (5 mg/kg/day), 14 days

Surgery and microdialysis experiments

Surgery was performed as previously described (Mørk, Eur. Neuropsychopharmacol. 1998, 8. p. 267-272). In brief, animals were anaesthetised with hypnorm/dormicum (2 ml/kg) and intracerebral guide cannulas (CMA/12) were stereotaxically implanted into the hippocampus, positioning the dialysis probe tip in the ventral hippocampus (co-ordinates: 5.6 mm posterior to bregma, lateral −4.8 mm, 4.0 mm ventral to dura) (Paxinos and Watson, 1986, The Rat Brain in Stereotaxic Coordinates, Academic Press, Inc. San Diego, USA). The rats were allowed to recover from surgery for at least 2 days, housed singly in cages.

On the day of the experiment, a microdialysis probe (CMA/12, 0.5 mm diameter, 3 mm length) was inserted through the guide cannula. Perfusion of the microdialysis probe with filtered Ringer solution (145 mM NaCl, 3 mM KCl, 1 mM MgCl₂, 1.2 mM CaCl₂) was begun shortly before insertion of the probe into the brain and continued for the duration of the experiment at constant flow of 1 μl/min. After the experiments, the rats were sacrificed by decapitation, their brains frozen and sliced (20 μm) and the position of the probes was verified.

Analysis of dialysate 5-HT

Concentration of 5-HT in the cortical dialysates was analysed by means of HPLC with electrochemical detection. Indoles were separated by reverse phase liquid chromatography (ODS 150×3 μm, 3 μm) using a mobile phase consisting of 50 mM NaH₂PO₄, 40 mg/l EDTA, 90 mg sodium octanesulfonic acid, and 9% acetonitrile (pH 3.5) at a flow rate of 0.5 ml/min. Electrochemical detection was accomplished using a coulometric detector, potential set at 220 mV (Coulochem II, ESA).

Results:

A single injection of paroxetine increased extracellular 5-HT in the ventral hippocampus by 200% (preinjection levels normalised to 100%). Injection of irindalone contemporarily with paroxetine significantly increased the effect of paroxetine (300%) (preinjection levels normalised to 100%) (P<0.05). Irindalone alone did not affect the extracellular level of 5-HT.

After 2 days of treatment, extracellular 5-HT was significantly increased in the ventral hippocampus (295%) in animals treated with the combination of paroxetine and irindalone, compared to levels in vehicle animals (normalised to 100%) and animals treated with paroxetine alone (138%)(P<0.05, group 3 vs group 1 and 2—no significant difference between group 1 and 2.

Paroxetine treatment for 14 days significantly increased extracellular 5-HT levels compared to the vehicle treatment (P<0.05, group 5 vs group 4). However, after 14 days of combined treatment with paroxetine and irindalone, extracellular 5-HT was significantly higher than that observed in the paroxetine group (P<0.05, group 6 vs group 5).

After 14 days of treatment with paroxetine alone a challenge dose of irindalone (1 mg/kg s.c.) significantly increased the extracellular 5-HT level (270%); prechallenge 5-HT levels normalised to 100% (P<0,001). Irindalone exerted no effect in vehicle animals.

Both citalopram and paroxetine have been demonstrated to increase baseline levels of extracellular 5-HT after 14 days of treatment. The effect of the combination of paroxetine and irindalone is observed after 2 days of treatment. These results show that the combination of paroxetine and irindalone provides a fast onset of action and that the potentiating effect of irindalone on paroxetine-induced increase in extracellular 5-HT is persistent following chronic administration.

Thus a better efficacy may be obtained by combined treatment with SRI and irindalone (add-on potential).

Schedule-induced polydipsia

Background:

In the schedule-induced polydipsia paradigm, food restricted rats are trained to take a food reward in an operant test chamber, the food reward is delivered on a fixed schedule and the rats exhibit displacement, or stress control, strategies between the delivery of the food pellets. In the present model the animals have free access to water and the rats drink significantly more than would be expected in a normal animal during the time allowed for the test. The anti-stress effects of chronic administration of serotonin reuptake inhibitors has be demonstrated: fluoxetine, clomipramine and fluvoxamine have each been demonstrated to reduce schedule-induced water intake, their effects are only observed following chronic administration (1-3 weeks; Woods et al. Psychopharmacology 1993, 112,195-198,). Further, the onset of action of action of the SSRI fluoxetine has been demonstrated to be accelerated by the 5-HT_{1A} and 5-HT_{1B} receptor antagonists, WAY 100,635 and GR 127935, respectively (Hogg and Arnt Society for Neuroscience 2000, vol 26, abstract No 721.16).

Materials and methods:

Rats (male, wistar WU from Mølleg [symbol] ard, Denmark) were maintained on a food restricted diet and maintained at 85-90% of their free feeding weight for at least 7 days prior to the start of testing. Animals were trained (4-5 times per week) in an operant chamber on a fixed schedule (1 pellet every 30s) for 30 minutes per session. Drinking water was freely available, water intake was measured for every trial session. Following three weeks of training water intake was stable (approximately 12 mL/rat/30 mm) and the animals were randomly allocated to treatment groups.

In the present experiment animals were tested on the first day with vehicle administration (all animals) and on days 2 and 3 of testing with one of the following treatments:

1. vehicle (paroxetine)+vehicle (irindalone)

2. vehicle (paroxetine)+5 mg/kg irindalone

3. 20 mg/kg paroxetine+vehicle (irindalone)

4. 20 mg/kg paroxetine+5 mg/kg irindalone

All treatments were p.o. 60 min prior to the start of the test sessions.

Water intake was calculated as % baseline (pretreatment) levels.

Results:

The vehicle treatments given on the first test day did not affect water intake. Treatment with vehicle, paroxetine or irindalone alone did not significantly affect the intake of water relative to baseline or vehicle treated animals; irindalone reduced intake be approximately 8% and paroxetine by appr. 12% relative to day one.

The combination of paroxetine and irindalone reduced water intake. A significant effect was observed after the first administration of the compounds whereby the water consumption was reduced to 50% of baseline levels (P<0.05 vs vehicle treated controls, paroxetine and irindalone alone).

The SRIs fluvoxamine, clomipramine and fluoxetine have all been demonstrated to reduce the intake of water in the schedule induce polydipsia model. The onset of action of the SSRIs alone is seen after 1-3 weeks, mirroring the clinical situation. The effect of the combination of paroxetine and irindalone is observed after one day's treatment suggesting that this combination would have a fast onset of effect in the clinic.

Light/dark box model in the mouse

Materials and methods:

Adult male NMRI mice were obtained from Mølleg [symbol] ard and housed in group cages (10 per cage) on a reversed light cycle (12 hour cycle, lights on 18:00) for 3 weeks prior to testing or the start of treatment. The two-compartment test box is described by Sanchez et al. Pharmacology & Toxicology 1995, 77, 71-78. The test box is open-topped and divided into one black and one white compartment (ratio 2:3) by a partition which is black on the side facing the black compartment and white on the side facing the white compartment. The white chamber is made of white perspex except for the lowest 7.5 cm. This part is made of transparent perspex (outer walls) and black perspex (partition). The floor of the white compartment is divided into 9 fields and the compartment is illuminated by means of a Schott KL 1500 electronic lamp emitting cold light (560 Lux). The black box is made of black perspex and the floor of this compartment is divided into 6 fields. The opening in the partition between the two compartments measures 7,5×7,5 cm.

The test system was fully automated by two rows of 11 infrared light sources and photocells in the transverse direction and one row of 16 photocells in the transverse direction (lower row). The lower row of photocells (2 cm above cage floor) detects horizontal activity (crossing, entries and time in each compartment), whereas the upper row of photocells (5 cm above cage floor) detects rearing activity.

The black and white boxes are placed in a dark room. The mice were transported to the animal holding room adjacent to the test room 18 hours before the test.

The test was performed as follows: the mouse was placed into the centre of the brightly-lit white compartment facing the opening to the black compartment and allowed to freely explore the apparatus for a total of 5 min. The light beams were used to assess activity around the test box and critically the time for which the animals explored the light and dark sections thereof.

Treatment:

Acute interaction experiments:

Animals were randomly assigned to the following groups (8 mice per group)

1. vehicle (paroxetine)+vehicle (irindalone)

2. paroxetine (5 mg/kg)+vehicle (irindalone)

3. paroxetine (vehicle)+irindalone (0.31 mg/kg)

4. paroxetine (vehicle)+irindalone (1.3 mg/kg)

5. paroxetine (5 mg/kg)+irindalone (0.31 mg/kg)

6. paroxetine (5 mg/kg)+irindalone (1.3 mg/kg)

Treatments were given subcutaneously 30 min prior to testing.

Result—acute interaction.

Animals in group 1 spent an average of 127 s (from a maximum of 300 s) in the light section of the box. Treatment with paroxetine (group 2) increased this to 143 s which is not significant. Animals treated with irindalone alone (groups 3 and 4) had average times in light of 140 and 148 s for 0.31 and 1.3 mg/kg doses, respectively, which is not significant. The mice treated with the combination of paroxetine and irindalone spent an average of 162 s (group 5) and 174 s (group 6) in the light. Group 5 was significantly different from group 1 (P<0.05) whilst group 6 was significantly different from group 1, group 2 and group 4 (P<0.05).

Thus animals treated with the combination of irindalone and paroxetine appear to be less anxious than those treated with either compound alone. A similar effect has been observed using citalopram as the SSRI of choice.

Treatment: Chronic studies

Animals were treated chronically with an SSRI and acutely with irindalone according to the following schedule:

| | Chronic treatment (28 days s.c. | Acute treatment (s.c. 30 min |
|---|---|---|
| Group | with minipumps) | prior to test) |
| 1 | Vehicle | Vehicle |
| 2 | 5 mg/kg/day | Vehicle |
| 3 | 10 mg/kg/day | Vehicle |
| 4 | 20 mg/kg/day | Vehicle |
| 5 | Vehicle | 1.3 mg/kg |
| 6 | 5 mg/kg/day | 1.3 mg/kg |
| 7 | 10 mg/kg/day | 1.3 mg/kg |
| 8 | 20 mg/kg/day | 1.3 mg/kg |

Results:

Animals treated with the two vehicles had an average time in light of 123±16 s. Treatment with paroxetine did not significantly change the level of exploration in light (group 2, 132±10 s; group 3, 140±12 s; group 4, 157±10 s; not significant). Similarly, irindalone alone (group 5) was without effect (average time in light 142±20 s; not significant). The animals treated with the combination of paroxetine and irindalone have the following average times in light; group 6, 143±11 s; group 7, 203±22 s (P<0.05 vs groups 1 and 3) and group 8; 206±10 s (P<0.05 vs groups 1, 4 and 5).

Thus the animals treated with the combination of chronic paroxetine and acute irindalone appeared less anxious than those treated with either compound alone.

A similar result has also been obtained using citalopram as the SSRI of choice.

Thus, irindalone potentiated the anxiolytic potential of paroxetine suggesting better efficacy for the combination treatment than the paroxetine alone. The benefit was observed following acute and importantly chronic treatment with paroxetine suggesting the potential of irindalone as an “add-on” therapy to enhance the efficacy of SSRIs.

1. The use of irindalone or a pharmaceutically acceptable salt thereof for the preparation of a pharmaceutical composition to be used in combination with a serotonin reuptake inhibitor inhibitor or any other compound, which causes an elevation in the level of extracellular serotonin.

2. The use of irindalone or a pharmaceutically acceptable salt thereof for the preparation of a pharmaceutical composition useful for augmenting and/or providing faster onset of the therapeutic effect of a serotonin reuptake inhibitor or any other compound, which causes an elevation in the level of extracellular serotonin.

3. The use according to claims 1 or 2 wherein the serotonin reuptake inhibitor or the compound, which causes an elevation in the level of extracellular serotonin, is used in the treatment of depression, anxiety disorders and other affective disorders, eating disorders such as bulimia, anorexia and obesity, phobias, dysthymia, premenstrual syndrome, cognitive disorders, impulse control disorders, attention deficit hyperactivity disorder and drug abuse.

4. The use according to claim 3 wherein the serotonin reuptake inhibitor or the compound, which causes an elevation in the level of extracellular serotonin, is used in the treatment of anxiety disorders including general anxiety disorder, panic anxiety, obsessive compulsive disorder, acute stress disorder, post trauma stress disorder or social anxiety disorder.

5. The use according to claim 3 wherein the serotonin reuptake inhibitor or the compound, which causes an elevation in the level of extracellular serotonin, is used in the treatment of depression.

6. The use according to claims 1 to 5 wherein the tartrate salt of irindalone is used.

7. The use according to claims 1 to 6 wherein the serotonin reuptake inhibitor is selected from citalopram, escitalopram, fluoxetine, sertraline, paroxetine, fluvoxamine, venlafaxine, dapoxetine, nefazodone, imipramin, femoxetine and clomipramine or a pharmaceutically acceptable salt of any of these compounds.

8. The use according to claim 7 wherein the serotonin reuptake inhibitor is a selective serotonin reuptake inhibitor.

9. The use according to claim 8 wherein the serotonin reuptake inhibitor is selected from citalopram, escitalopram, fluoxetine, fluvoxamine, sertraline and paroxetine or a pharmaceutically acceptable salt of any of these compounds.

10. The use of irindalone or a pharmaceutically acceptable salt thereof and a compound, which is a serotonin reuptake inhibitor, or any other compound which causes an elevation in the level of extracellular serotonin, for the preparation of a pharmaceutical composition or kit for the treatment of diseases or disorders responsive to the therapeutic effect of a serotonin reuptake inhibitor or any other compound causing an elevation in the level of extracellular serotonin.

11. The use according to claim 10 for the preparation of a pharmaceutical composition or kit useful for the treatment of depression, anxiety disorders and other affective disorders, eating disorders such as bulimia, anorexia and obesity, phobias, dysthymia, premenstrual syndrome, cognitive disorders, impulse control disorders, attention deficit hyperactivity disorder and drug abuse.

12. The use according to claim 11 for the preparation of a pharmaceutical composition or kit useful for the treatment of anxiety disorders, including general anxiety disorder, panic anxiety, obsessive compulsive disorder, acute stress disorder, post trauma stress disorder or social anxiety disorder.

13. The use according to claim 11 for the preparation of a pharmaceutical composition for the treatment of depression.

14. The use according to claims 10 to 13 wherein irindalone is used in the form of a tartrate salt.

15. The use according to claims 10 to 14 wherein the serotonin uptake inhibitor is selected from citalopram, escitalopram, fluoxetine, sertraline, paroxetine, fluvoxamine, venlafaxine, dapoxetine, nefazodone, imipramin, femoxetine and clomipramine or a pharmaceutically acceptable salt of any of these compounds.

16. The use according to claim 15 wherein the serotonin reuptake inhibitor used is a selective serotonin reuptake inhibitor.

17. The use according to claim 16 wherein the selective serotonin reuptake inhibitor is citalopram, escitalopram, fluoxetine, fluvoxamine, sertraline and paroxetine or a pharmaceutically acceptable salt of any of these compounds.

18. The use according to claims 10-17 wherein the pharmaceutical composition prepared is adapted for simultaneous administration of the active ingredients.

19. The use according to claim 18 wherein the active ingredients are contained in the same unit dosage form.

20. The use according to claims 10-17 wherein the pharmaceutical composition prepared is adapted for sequential administration of the active ingredients.

21. The use according to claim 20 wherein the active ingredients are contained in discrete unit dosage forms.

22. A pharmaceutical composition or kit comprising irindalone or a pharmaceutically acceptable salt thereof and a compound, which is a serotonin reuptake inhibitor, or any other compound which causes an elevation in the level of extracellular serotonin, and optionally pharmaceutically acceptable carriers or diluents.

23. A pharmaceutical composition or kit according to claim 22 containing irindalone in the form of the tartrate salt thereof.

24. The pharmaceutical composition or kit according to claims 22 to 23 characterised in that the serotonin uptake inhibitor is selected from citalopram, escitalopram, fluoxetine, sertraline, paroxetine, fluvoxamine, venlafaxine, dapoxetine, nefazodone, imipramin, femoxetine and clomipramine or a pharmaceutically acceptable salt of any of these compounds.

25. The pharmaceutical composition or kit according to claim 24 wherein the serotonin reuptake inhibitor is a selective serotonin reuptake inhibitor.

26. The pharmaceutical composition or kit according to claim 25 wherein the selective serotonin reuptake inhibitor is citalopram, escitalopram, fluoxetine, fluvoxamine, sertraline and paroxetine or a pharmaceutically acceptable salt of any of these compounds.

27. The pharmaceutical composition according to claims 22 to 26 which is adapted for simultaneous administration of the active ingredients.

28. The pharmaceutical composition according to claim 27 wherein the active ingredients are contained in the same unit dosage form.

29. The pharmaceutical composition or kit according to claims 22 to 26 which is adapted for sequential administration of the active ingredients.

30. The pharmaceutical composition according to claim 29 wherein the active ingredients are contained in discrete dosage forms.

31. A method for the treatment of diseases or disorders responsive to a serotonin reuptake inhibitor or any other compound which causes an elevation in the level of extracellular serotonin, comprising administering irindalone or a pharmaceutically acceptable salt thereof and a serotonin reuptake inhibitor, or a compound which causes an elevation in the level extracellular serotonin, to an individual in need thereof.

32. A method for augmenting and/or providing faster onset of the therapeutic effect of a serotonin reuptake inhibitor, or any other compound which causes an elevation in the level of extracellular serotonin, comprising administering irindalone or a pharmaceutically acceptable salt thereof to an individual to be treated with or undergoing treatment with the serotonin reuptake inhibitor, or any other compound which causes an elevation in the level of extracellular serotonin.

33. A method according to claims 31 to 32 wherein the individual suffers from depression, anxiety disorders and other affective disorders, eating disorder; such as bulimia, anorexia and obesity, phobias, dysthymia, premenstrual syndrome, cognitive disorders, impulse control disorders, attention deficit hyperactivity disorder and drug abuse.

34. The method according to claim 33 wherein the individual suffers from anxiety disorder, including general anxiety disorder, panic anxiety, obsessive compulsive disorder, acute stress disorder, post trauma stress disorder or social anxiety disorder.

35. The method according to claim 33 wherein the individual suffers from depression.

36. The method according to claims 31 to 32 wherein irindalone is used in the form of the tartrate salt.

37. The method according to claims 31 to 32 wherein the serotonin uptake inhibitor is selected from citalopram, escitalopram, fluoxetine, sertraline, paroxetine, fluvoxamine, venlafaxine, dapoxetine, nefazodone, imipramin, femoxetine and clomipramine or a pharmaceutically acceptable salt of any of these compounds.

38. The method according to claim 37 wherein the serotonin reuptake inhibitor is a selective serotonin reuptake inhibitor.

39. The pharmaceutical composition according to claim 38 wherein the selective serotonin reuptake inhibitor is citalopram, escitalopram, fluoxetine, fluvoxamine, sertraline and paroxetine or a pharmaceutically acceptable salt of any of these compounds.

40. The method according to claims 31-39 wherein the active ingredients are administered simultaneously.

41. The method according to claim 40 wherein the active ingredients are administered in the same unit dosage from.

42. The method according to claims 31-39 wherein the active ingredients are administered sequentially.

43. The method according to claim 42 wherein the active ingredients are contained in two discrete unit dosage forms.
